(19) United States
(12) Patent Application Publication
(10) Pub. No.: US 2002/0137926 A1
(43) Pub. Date: Sep. 26, 2002

(54) Process for preparing crystalline salts of amoxycillin

(75) Inventors: Esin F. Kosal, Holmdel, NJ (US); Paul G. Butterly, Clark, NJ (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/074,824

(22) Filed: Feb. 13, 2002

Related U.S. Application Data
(63) Continuation of application No. 09/763,569, filed on Nov. 28, 2000, now abandoned.
Application No. 09/763,569 is a 371 of international application No. PCT/US99/11991, filed on Jun. 1, 1999.
(60) Provisional application No. 60/087,554, filed on Jun. 1, 1998.

(51) Int. Cl. C07D 499/16

(52) U.S. Cl. 540/321

(57) ABSTRACT

A novel process for preparing a crystalline alkali metal salt of amoxycillin is dislcosed.

This application claims the benefit of U.S. Provisional Application No. 60/087,554, filed Jun. 1, 1998.

This invention relates to a process for the preparation of crystalline salts of the beta lactam antibiotic amoxycillin viz. 6-[[amino (4-hydroxyphenyl)acetyl]-amino]-3,3-dimethyl-7-oxo-4-thia-1-azabicyclo [3.2.0] heptane-2-carboxylic acid. In particular this invention relates to a process for the preparation of crystalline sodium amoxycillin.

Crystalline sodium amoxycillin is a known substance and processes for its preparation are disclosed in the state of the art. For example EP 0131147A discloses a process in which amoxycillin trihydrate is converted into a crystalline solvate of sodium amoxycillin from which the solvating solvent is removed. In one experimental example of this disclosure amoxycillin trihydrate is suspended in methyl acetate, then a solution of a mixture of triethylamine and sodium 2-ethylhexanoate is added to this suspension. In EP 0596262A amoxycillin trihydrate is dissolved in a methyl acetate/isopropanol/triethylamine mixture, then this solution is added to a solution of sodium 2-ethylhexanoate in a methyl acetate/methanol mixture. A crystalline solvate of sodium amoxycillin is believed to crystallize out from the reaction mixture, from which the solvating solvent is removed. In WO 97/15579 amoxycillin trihydrate is added to an ethanol/triethylamine mixture to form a solution which is then reacted with sodium 2-ethylhexanoate in ethanolic solution, and a crystalline product is obtained. In U.S. Pat. No. 4,737,585 amoxycillin trihydrate is suspended in a mixture of an aprotic solvent, such as methylene chloride, and a lower alcohol, the amoxycillin is solubilized using a low molecular weight amine, and to this mixture is added the sodium salt of diethyloxalacetic acid. Sodium amoxycillin is then precipitated by addition of more of the aprotic solvent.

In processes used to prepare crystalline sodium amoxycillin it is desirable to minimize the quantities of solvents used and to improve yield and purity of the product. Consequently there is an ongoing problem of process improvement. It is an object of the present invention to provide an alternative and improved process for the preparation of crystalline sodium amoxycillin. Other objects and advantages of the present invention will be apparent from the following description.

According to this invention a process for the preparation of a crystalline alkali metal salt of amoxycillin is provided, in which;

a suspension of an amine salt of amoxycillin is formed in a first organic solvent,

the suspension is admixed with a second organic solvent and the amine salt is caused to enter solution in the so-formed mixture of first and second organic solvents,

the amine salt is reacted with a salifying compound of the alkali metal,

the so-formed alkali metal salt of amoxycillin is isolated from the solution as a crystalline product.

In the process of the invention it is believed that the formation of the suspension of the amine salt in the first solvent, and rapid dissolution of the salt when the second organic solvent 1-5is admixed, leads to decreased breakdown of the amoxycillin, and consequently increased yield and purity of the crystalline product. In the process of this invention the reagents are formed into solution, which facilitates sterilising filtration and consequent use of the product as an injectible pharmaceutical product. However the process of the present invention may equally effectively be used without a sterile filtration step to prepare crystalline sodium amoxycillin which may be administered orally.

Preferably the crystalline alkali metal salt of amoxycillin prepared by the process of this invention is crystalline sodium amoxycillin.

Preferably the amine salt of amoxycillin is a salt of a tri- or di-(C1-51-5)alkyl amine, such as triethylamine, diethylamine or diisopropylamine, especially the triethylamine, salt of amoxycillin. A mixture of amine salts may be used such as a mixture of the salts of amoxycillin with triethylamine and diisopropylamine. Other suitable amine salts include the salt with dicyclohexylamine.

A preferred first organic solvent is a (C1-51-5)alkyl (C1-51-5)alkanoate ester, a preferred such ester being a (C1-51-5)alkyl acetate ester, particularly methyl acetate. The first organic solvent may comprise a single solvent or a mixture of solvents, for example a mixture of said esters or said esters and other co-solvents.

Preferably the suspension is formed by first forming a suspension of amoxycillin, preferably in the form of amoxycillin trihydrate, in the first organic solvent then admixing the amine with this suspension so that the amine reacts with the amoxycillin to form the amine salt. This reaction is preferably carried out at below ambient temperature, for example below 10° C., especially at 0-5° C. Suitably the amoxycillin trihydrate may be suspended in a volume of methyl acetate at a ratio weight of amoxycillin trihydrate:volume of methyl acetate ca. 1:1-1:2.5, for example typically 1:1.7-1:2, and this suspension may be admixed with the triethylamine in a stoichiometric excess to the amoxycillin, for example at a molar ratio amoxycillin:triethylamine 1:1-1:2, for example typically 1:1.3-1:1.5.

A suitable second organic solvent with which the suspension of the amine salt may be admixed is a (C1-51-5)alcohol, such as methyl alcohol, which is preferred, or ethyl alcohol, propyl alcohol e.g. isopropyl alcohol, or butyl alcohol e.g. iso-butyl alcohol. The second organic solvent may comprise a single solvent or a mixture of solvents, for example a mixture of said alcohols or said alcohols and other co-solvents.

The admixing of the second solvent and the suspension of the amine salt in the first solvent causes the salt to enter solution, and the volume of the second solvent e.g. the alcohol used may be determined experimentally as the minimum volume of the second solvent necessary to achieve this. Typically when the first solvent is the above-described ester and the second solvent is the above-described alcohol a volume ratio ester:alcohol of ca. 1:0.3-0.6 will be found suitable, e.g. about 1:0.4-0.5 of methyl acetate:methyl alcohol. If such a volume ratio of methyl alcohol is admixed with the above-described suspension of the triethylamine salt of amoxycillin then the salt generally dissolves immediately upon stirring.

At this stage the so-formed solution of the amine salt of amoxycillin may for example be filtered and/or treated by other standard purification steps e.g. treatment with dicalite or other materials which selectively absorb impurities. If the solution is filtered the filter medium may subsequently be washed with more of the second solvent e.g. the alcohol, e.g. corresponding to an amount 0.5-1.0 of the amount already in admixture with the solution of the salt.

A suitable salifying compound is a pharmaceutically acceptable salifying compound of a suitable alkali metal, for example the sodium salt of an organic compound, for example an alcoholate such as of a (C1-51-5)alcohol e.g. the methoxide and/or ethoxide, or a salt of an organic acid e.g. a (C1-121-5) carboxylic acid such as an alkyl substituted-alkanoate for example a 2-ethylhexanoate. In the case of preparation of crystalline sodium amoxycillin sodium 2-ethylhexanoate is a preferred salifying compound.

The reaction of the amine salt is suitably carried out by admixing the solution of the amine salt with a solution of the salifying compound. Suitably the salifying compound is in solution in a solvent mixture which comprises the abovementioned first and second organic solvents, e.g. the abovementioned (C1-51-5)alkyl (C1-51-5)alkanoate ester and (C1-51-5)alcohol, for example a methyl acetate:methanol mixture comprising predominantly the ester, for example a 9-12:1 v:v, e.g. 10-11:1 v:v methyl acetate:methanol mixture. Suitably a stoichiometric excess of the salifying compound relative to the amoxycillin is used, e.g. a 1.5-2.5:1 molar ratio of sodium 2-ethyl hexanoate:amoxycillin. Suitably a solution of sodium 2-ethylhexanoate in the abovementioned solvent mixture which is of concentration around 1.8-2.5 M may be used. This solution may be filtered and/or subjected to other appropriate purification steps before the reaction with the amoxycillin.

Preferably the reaction between the salifying compound and the amoxycillin is carried out at below ambient temperature, e.g. below 10° C., especially at 0-5° C. Preferably the solution of the amine salt is added to the solution of the salifying compound, although the reverse mode of addition, i.e. addition of the solution of the salifying compound to the solution of the amine salt, or concurrent admixing, may be used. Preferably the mixing of the two solutions is carried out as fast as possible, with rapid stirring.

Spontaneous crystallization of the reaction product may occur, but it is preferred to induce crystallization by the addition of seed crystals, e.g. of crystalline sodium amoxycillin or some crystallographically equivalent material e.g. a solvate of crystalline sodium amoxycillin, to the reaction mixture immediately after the mixing of the solutions. To further encourage crystallization further of the abovementioned first organic solvent e.g. the (C1-51-5)alkyl (C1-51-5)alkanoate ester, e.g. methyl acetate, may be admixed with the reaction mixture, preferably in excess of the reaction medium volume e.g. in a 1.5-2.0× excess of the reaction medium volume. This admixing of the first organic solvent may be carried out relatively slowly, e.g. over a period of ca. 30-40 minutes. After the admixing of this further amount of the first solvent the mixture may be stirred for a time, e.g. ca. 1 hour preferably below ambient temperature, e.g. below 10° C., especially at 0-5° C.

After this stage the crystalline product may be separated off from the reaction medium using standard procedures, e.g. filtered off, and washed with a suitable wash liquid, preferably the first organic solvent.

The crystalline product obtained in this way is believed to be a crystalline solvate of sodium amoxycillin, e.g. the methyl acetate solvate from which the solvating solvent may be removed by a drying process, e.g. the process disclosed in EP 0131147A, the contents of which are incorporated herein by reference. This drying process may be in vacuum preferably at an elevated temperature such as 50-65° C., for example 60-65° C. to remove residual reaction medium solvents, wash liquids and solvating solvents, to yield the crystalline alkali metal salt of amoxycillin.

Crystalline sodium amoxycillin prepared by the process of this invention may be used as a pharmaceutical antibiotic product e.g. in injectible form. For this purpose it may be provided contained in sealed sterile vials. Alternatively the sodium amoxycillin prepared by the process of this invention may be used in formulations for oral administration e.g. in tablet, granule, syrup etc formulations and for oral administration sterile formulation is not necessarily required. Preferably for use as an antibiotic product the product crystalline alkali metal salt of amoxycillin is administered in combination with a pharmaceutically acceptable beta-lactamase inhibitor such as a salt of clavulanic acid, particularly potassium clavulanate.

The invention will now be illustrated by way of non-limiting example.

### EXAMPLE 1

### Laboratory Scale

1.1 Dissolution of Sodium 2-ethyl Hexanoate.

Sodium 2-ethyl hexanoate (160 g) was dissolved in a mixture of methyl acetate (310 ml) and methanol (30 ml). The mixture was stirred at room temperature until dissolution was complete and the solution was filtered through Whatman No. 1 filter paper to remove cloudiness. The solution was transferred to a crystallization vessel, followed by a wash of methyl acetate (210 ml) and stirred at 0-5° C. This solution was consequently 2.15M in Sodium 2-ethyl hexanoate.

1.2 Dissolution of Amoxycillin Trihydrate.

Amoxycillin trihydrate (250 g) was slurried in methyl acetate (450 ml). Triethylamine (120 ml) was added to the slurry creating a thick suspension. Methanol (200 ml) was added, causing the amoxycillin triethylamine salt to dissolve instantly. The solution was stirred for 5 minutes before dicalite (10 g) was added and the mixture was allowed to stir for a further 5 minutes. The solution was then filtered through Whatman No. 1 filter paper followed by a wash with methanol (120 ml) and transferred to the crystallization vessel containing sodium 2-ethyl hexanoate from 1.1 above.

1.3 Reaction and Crystallization.

The mixture in the crystallization vessel was stirred vigorously at 0-5° C. and crystalline sodium amoxycillin seed (1 g) was added. As crystallization began methyl acetate (2300 ml) was added over a period of 30-40 minutes. The mixture was allowed to stir for 1 hour at 0-5° C. The product was then filtered and washed with methyl acetate (750 ml).

1.4 Drying

The crystalline product from 1.3 was dried under vacuum at 50-55° C. for 36 hours, or alternatively at 65° C. for the shorter period of 16 hours. Product quality was found to be unaffected by the drying temperature. The product was confirmed to be crystalline sodium amoxycillin by chemical analysis, infrared spectroscopy and X-ray powder diffractometry.

The product obtained had an amoxycillin content of 91.0%, with a total content of impurities of 2.13-2.16%.

### EXAMPLE 2

### Scale Up

2.1 Dissolution of Sodium 2-ethyl Hexanoate.

Methyl acetate (124L) was added to sodium 2-ethyl hexanoate (64 kg), and methanol (12L) was added with stirring until the sodium 2-ethyl hexanoate dissolved. The solution was filtered and transferred to a crystallization vessel, washing in with methyl acetate (84L).

2.2 Dissolution of Amoxycillin Trihydrate.

Methyl acetate (124L) was added to amoxycillin trihydrate (87.1 kg) and the resulting suspension was cooled to 0-5° C. Triethylamine (48.1L) was added to the slurry and the slurry was stirred for 5 minutes. Methanol (80L) was added causing the amoxycillin triethylamine salt to dissolve and the solution was stirred for 5 minutes. Dicalite (2 kg) was added and the mixture was stirred for a further 5 minutes. The solution was then filtered and the cake was washed through with methanol (48L).

2.3 Reaction and Crystallization.

The solution of amoxycillin triethylamine salt from 2.2 was added to the sodium 2-ethyl hexanoate solution from 2.1 as fast as possible and the mixture was cooled to 0-5° C. Crystalline sodium amoxycillin seed (400 g) was added. Methyl acetate (921L) was added at a rate of 27 liters per minute. The mixture was stirred for 1 hour at 0-5° C. The slurry was then transferred to a Nutrex™ mixer and the mother liquor was filtered. The product was then washed with methyl acetate (84L).

2.4 Drying

The crystalline product from 2.3 was blow dried with nitrogen, and then dried in vacuum at 60-65° C.

The product obtained had an amoxycillin content of 89.7-93.6%, with a total content of impurities of 1.72-1.42% over three batches.

1. A process for the preparation of a crystalline alkali metal salt of amoxycillin is provided, in which;
a suspension of an amine salt of amoxycillin is formed in a first organic solvent,
the suspension is admixed with a second organic solvent and the amine salt is caused to enter solution in the so-formed mixture of first and second organic solvents,
the amine salt is reacted with a salifying compound of the alkali metal,
the so-formed alkali metal salt of amoxycillin is isolated from the solution as a crystalline product.

2. A process according to claim 1 wherein the crystalline alkali metal salt of amoxycillin prepared by the process is crystalline sodium amoxycillin.

3. A process according to claim 1 wherein the amine salt of amoxycillin is a triethylamine, diethylamine or diisopropylamine salt of amoxycillin.

4. A process according to claim 1 wherein the first organic solvent is a (C1-51-5)alkyl (C1-51-5)alkanoate ester

5. A process according to claim 4 wherein the (C1-51-5) alkyl (C1-51-5)alkanoate ester is methyl acetate.

6. A process according to claim 1 wherein the suspension of the amine salt is formed by first forming a suspension of amoxycillin trihydrate in the first solvent then admixing the amine with this suspension so that the amine reacts with the amoxycillin to form the amine salt.

7. A process according to claim 1 wherein the second organic solvent is a (C1-51-5)alcohol.

8. A process according to claim 7 wherein the (C1-51-5)alcohol is methyl alcohol.

9. A process according to claim 1 wherein the salifying compound is a sodium salt of an organic compound.

10. A process according to claim 9 wherein the salifying compound is a sodium salt of a (C1-51-5)alcohol or a salt of a (C1-121-5) carboxylic acid.

11. A process according to claim 8 wherein the salifying compound is sodium 2-ethylhexanoate.

12. A process according to claim 1 wherein the reaction of the amine salt is carried out by admixing the solution of the amine salt with a solution of the salifying compound.

13. A process according to claim 10 wherein the salifying compound is in solution in a solvent mixture which comprises the first and second organic solvents.

14. A process according to claim 13 wherein the solvent mixture is a methyl acetate:methanol mixture comprising predominantly methyl acetate.

15. A process according to claim 12 wherein the solution of the amine salt is added to the solution of the salifying compound.

16. A crystalline alkali metal salt of amoxycillin being the product of a process according to any one of the preceding claims.
